(19) United States
(12) Patent Application Publication
(10) Pub. No.: US 2005/0222082 A1
(43) Pub. Date: Oct. 6, 2005

(54) Agent for producing a sensation of safety and for weight loss

(76) Inventor: Gunther Beisel, Monheim (DE)

Correspondence Address: FRIEDRICH KUEFFNER, 317 MADISON AVENUE, SUITE 910, NEW YORK, NY, 10017, US

(21) Appl. No.: 10/511,518

(22) PCT Filed: Apr. 15, 2003
(86) PCT No.: PCT/EP03/03910

§ 371 Date: May 9, 2005

(30) Foreign Application Priority Data
Apr. 15, 2002 (DE) 10216551.3
Apr. 15, 2002 (DE) 20205854.9

(51) Int. Cl. A61K 31/732

(52) U.S. Cl. 514/54

(57) ABSTRACT

The present invention pertains to an improved agent for producing a satiety effect and for weight loss. The agent according to the invention is also suitable for regulating the cholesterol balance.

The present invention pertains to an agent for producing a satiety effect and for weight loss. The inventive agent is also suitable for regulating the cholesterol balance.

Numerous attempts have been made to help people decrease superfluous deposits of fat and to prevent them from accumulating by the use of medications. There are so-called appetite suppressants, for example, which attempt to suggest an aversion to eating by biochemical means. Some of these agents have considerable negative side effects.

In addition to numerous known dietary schemes, there are also mechanical and electromechanical agents which are claimed to have the ability effectively to eliminate fat and/or to build up muscle. The effects of these agents, however, are highly doubtful.

An agent for oral administration is known from DE 40 25 912, which consists of a container, which dissolves in the stomach and releases its content. This container is filled with a substance which, after being released in the stomach, increases in volume and thus suggests to the body that the stomach is full. The disadvantage of this satiety agent is that it is associated with the danger of intestinal blockage.

Sponge-like preparations with stably crosslinked compounds are known from DE 199 42 417. These preparations expand in volume in the stomach and thus produce a feeling of satiety. The production of these preparations requires additional process steps, however, to introduce the stable crosslinking.

Because of a steady increase in health awareness, however, further improvement of the agents available so far for producing a satiety effect is of high medical and economic relevance.

The task of the present invention is to provide an improved agent for oral administration, which remains for a longer time in the stomach than known agents of its type and thus leads to a more effective satiety effect. In addition, the agent is also intended to be suitable for weight loss. Its suitability for regulating the cholesterol level should also prove to be advantageous, because excess weight is usually associated with elevated cholesterol levels. Its ease of production from inexpensive raw materials which conceal no health risks should also be desirable.

The present task is accomplished by an agent for producing a satiety effect and for weight loss consisting of the dried, porous gel or foam of at least one anionic polymer, where the gel or the foam is present as an aluminum salt.

Preferred anionic polymers according to the invention are polysaccharides and polyuronic acid-containing polysaccharides such as alginic acid and its salts (alginates). Low-esterified pectins, xanthan gum, gum tragacanth, chondroitin sulfate, and any other uronic acid-containing compounds, however, can also be used according to the invention. The use of synthetic or semi-synthetic cellulose derivatives such as carboxymethylcellulose or of polyacrylates can also be considered.

Dried gels or foams containing mixtures of anionic polymers, preferably of the previously mentioned anionic polysaccharides, more preferably mixtures of polyuronic acid-containing and low-esterified polysaccharides, and especially mixtures which contain salts of alginic acid and pectin are advantageous according to the invention.

Alginic acid is a linear polyuronic acid consisting of varying fractions of D-mannuronic acid and L-guluronic acid, which are linked to each other by β-glycoside bonds. The carboxyl groups are not esterified. One molecule of alginic acid can be built up out of approximately 150-1,050 uronic acid units, and the average molecular weight can vary within the range of 30-200 kDa.

The polysaccharide alginic acid is a constituent of the cell walls of brown algae. The alginic acid can constitute up to 40% of the dry weight of the algae. Alginic acid is recovered from algae by alkaline extraction with the use of known methods according to the state of the art. The resulting alginic acid powder is thus purely of plant origin and has a high degree of biocompatibility. It can absorb 300 times its own weight in water to form highly viscous solutions. In the presence of polyvalent cations, alginic acid forms so-called gels. The formation of alginate gels in the presence of divalent cations such as calcium or barium is described by I. Shapiro et al. (Biomaterials, 1997, 18: 583-90). Because of its toxicity, however, barium is not suitable for use in biomedicine. In addition to calcium chloride, calcium gluconate also supplies suitable divalent cations. The use of magnesium salts or mixtures of various physiologically safe divalent cations is also conceivable.

With respect to the anionic polymers, the use of low-esterified pectins is also advantageous according to the invention. Pectins consist of chains of galacturonic acid units linked together by [symbol] 1,4-glycosidic linkages, 20-80% of the acid groups of which are esterified with methanol. A distinction is made between high-esterified (>50%) and low-esterified (<50%) pectins. The molecular weight varies between 10 and 500 kDa. Pectins are recovered by acid extraction according to state-of-the-art methods known in themselves from the inner portions of citrus fruit peels, fruit residues, and sugar beet chips. The resulting pectins (apple pectin, citrus pectin) are thus purely of plant origin and have a high degree of biocompatibility. They can absorb water to form gels.

Here, too, the use of pectin gels in the presence of divalent cations such as calcium or barium is already known. Again, because it is toxic, barium is not suitable for use in biomedicine. In addition to calcium chloride, calcium gluconate also supplies suitable divalent cations. The use of magnesium salts or mixtures of various physiologically safe divalent cations is also conceivable.

The use according to the invention of pectins is also advantageously characterized in that pectins have cholesterol-lowering properties. This property is advantageous in the inventive sense because excessive body weight is usually associated with elevated cholesterol levels.

Processes for the production of dry gels or dry foams from alginates are known. A solution of sodium alginate in water, for example, is first produced and then thickened by the addition of calcium salts. By the incorporation of air and possibly the addition of surfactants, a gel or a foam can be obtained. By freezing and then freeze-drying, a dry gel or dry foam (sponge) is then obtained from the alginate gel or foam. Pectin-containing gels or foams are produced in the same way, as are gels or foams containing mixtures of anionic polymers.

In addition to the use of inorganic or organic calcium salts such as calcium chloride and calcium gluconate, magnesium salts can also be considered as well as mixtures of various physiologically safe divalent cations.

The addition of salts of physiologically safe trivalent cations, preferably soluble aluminum salts, is especially preferred according to the invention. The agents according to the invention can be produced by the addition of soluble aluminum salts to an aqueous solution of anionic polymers, preferably alginates and/or pectins, according to a production process of the previously described type. Especially preferred soluble aluminum salts are aluminum chloride and aluminum sulfate. The soluble aluminum salts can be used alone or in combination. According to the invention, salts of divalent cations such as calcium or magnesium salts or combinations of them can also be used in addition to the soluble aluminum salts, which can for their own part be used alone or in combination, for the production of the inventive agents.

The object of the present invention is therefore also a process for the production of an improved agent for achieving a satiety effect or for weight loss, according to which process water-soluble salts containing trivalent cations, preferably aluminum salts, even more preferably aluminum chloride or aluminum sulfate, are used for the production of a dried gel or foam of at least one anionic polymer. In addition, salts of physiologically safe divalent cations can also be used as well as conceivable combinations of salts of divalent and/or trivalent cations. The use of anionic polymers individually or in combination is also included in the invention.

The inventive agent for oral administration contains at least one anionic polymer in the form of its aluminum salt. The inventive agent advantageously contains alginate or pectin or a combination thereof as its anionic polymer. The inventive agent is preferably in the form of aluminum alginate or aluminum pectin or a mixture of aluminum alginate and aluminum pectin.

The salt of trivalent cations, preferably in the form of an aluminum salt, reacts with the anionic polymers, preferably alginates or pectins, to form complexes which are more stable than those obtained with the previously used salts of divalent cations. Aluminum, furthermore, is physiologically safe, in contrast to barium. The more stable interaction of the inventive anionic polymers with salts of trivalent cations awards the inventive agent the advantageous properties, first, of being insoluble or only sparingly soluble in solutions with a pH of 1-5, preferably in solutions with a pH of 1-4, and even more preferably in solutions with a pH comparable to that of the stomach or in the stomach itself; and, second, of dissolving completely in neutral-to-weakly acid solutions with a pH value of about 6-7, preferably in solutions with a pH comparable to that of the intestine or in the intestine itself. The dissolution of the inventive agent containing aluminum alginates begins, for example, at a pK value of approximately 3.3-3.7.

In addition to the previously described behavior of the inventive agents with respect to their solubility, the agents also have the advantageous property of increased dimensional stability. This dimensional stability is especially pronounced in the case of agents which contain mixtures of anionic polymers in the form of their aluminum salts, preferably mixtures of aluminum alginate and aluminum pectinate. The term “dimensional stability” according to the invention is understood to mean that the inventive agents containing aluminum salts of anionic polymers do not shrink in solutions with a pH of approximately 1-5, in contrast to gels or foams which contain calcium salts of anionic polymers alone. That is, known agents consisting of calcium salts of anionic polymers suffer from the disadvantage that they lose at least one-third of their volume in acid solutions, usually even more. The advantage of the dimensional stability of the inventive agents therefore has a direct, positive effect on their ability to produce a feeling of satiety or to help weight loss, because the inventive agent does not lose volume when it enters the stomach, which cannot be said for the agents known so far. According to the invention, therefore, it is not necessary to increase the amount of satiety agent ingested to compensate for the loss of volume. This represents a welcome side effect for the consumer.

The inventive agent in the form of a gel or foam, furthermore, is preferably present in compressed form when being ingested by the patient. In another embodiment, the inventive agent can also be compressed during ingestion by chewing and/or swallowing movements. As a result of the uptake of fluid in the stomach, the ingested inventive agent expands in volume, which triggers the desired effect of producing a feeling of satiety, which can lead in turn to a loss of weight.

The inventive agent, furthermore, can be present, for example, in the form of tablets, capsules, coated tablets, granulates, powders, or other forms. The inventive agent, furthermore, can also have a coating as an outer layer. In a variant of the inventive production method, an outer layer, referred to as a coating, which contains additional auxiliary substances or active ingredients can be applied to the inventive agent. The additional substances can be, for example, compounds which make it easier for the inventive agent to be swallowed or ingested and which are known to the expert as “coating compounds”. This outer layer can be a lacquer layer or some other type of protective layer, which makes it easier to ingest the inventive agent and which does not dissolve until it reaches the gastrointestinal tract, where it is exposed to the influence of, for example, gastric juice.

The inventive agent can also contain additional auxiliary substances and/or active ingredients.

The term “auxiliary substances” is understood to include, for example, the following substances, but the list is not to be considered a limitation on the present invention: water-insoluble auxiliary substances or mixtures thereof such as lipids, including fatty alcohols such as cetyl alcohol, stearyl alcohol, and cetostearyl alcohol; glycerides such as glycerol monostearate or mixtures of mono-, di-, and triglycerides of vegetable oils; hydrogenated oils such as hydrogenated castor oil and hydrogenated cottonseed oil; waxes such as beeswax and carnauba wax; solid hydrocarbons such as paraffin or mineral wax; fatty acids such as stearic acid; certain cellulose derivatives such as ethylcellulose and acetyl cellulose; polymers or copolymers such as polyalkylenes (e.g., polyethylene), polyvinyl compounds (e.g., polyvinyl chloride and polyvinyl acetate), vinyl chloride-vinyl acetate copolymers and copolymers with crotonic acid, and polymers and copolymers of acrylates and methacrylates (e.g., copolymers of acrylic acid ester and methacrylic acid methyl ester); and surfactants such as Polysorbate 80 and docusate.

“Active ingredients” are understood to include, for example, vitamins, trace elements, and medicinal compounds. The following substances can be listed by way of example and not to be considered limitations on the present invention:

Examples of appetite suppressants are: amfepramon, fenfluramine, fenproporex, levopropylhexedrine, mazindole, mefenorex, metamfepramone, norephedrine, norpseudoephedrine.

Examples of virustatics are: acyclovir, cidofovir, didanosine, famcyclovir, foscarnet, ganciclovir, lamivudine, ritonavir, zalcitabine, zidovudine.

Examples of vitamins are: alfacalcidol, allithiamin, ascorbic acid, biotin, calcifediol, calcitriol, cholecalciferol, cyanocobalamin, ergocalciferol, folic acid, hydrocobalamine, nicotinamide, pantothenic acid, phytomenadione, pyridoxine, retinol, riboflavin, thiamine, tocopherol, and trans-calcifediol.

Under certain conditions, the release of the active ingredient can also be delayed.

In addition to the cited auxiliary substances and active ingredients, the inventive agent can also contain fillers, disintegration agents, binders, and lubricants as well as carriers with no significant effect on the release of the active ingredient. Examples include bentonite (hydrate of aluminum oxide and silicon oxide), silicic acid, cellulose (usually microcrystalline cellulose) and cellulose derivatives such as methylcellulose and sodium carboxymethylcellulose, sugars such as lactose, starches such cornstarch or derivatives thereof such as sodium carboxymethyl starch, starch paste, phosphoric acid salts such as di- and tricalcium phosphate, gelatins, stearic acid and suitable salts thereof such as magnesium stearate and calcium stearate, talc, colloidal silicon oxide, and similar auxiliary materials.

The present invention also pertains to the use of the inventive agent for producing a satiety effect and for weight loss. The use of the inventive agent for regulating the cholesterol balance is also included.

The use of the inventive agent for the production of a composition for producing a satiety effect and for weight loss is also conceivable. Similarly, the use of the inventive agent for producing a composition for regulating the cholesterol level is also included.

The present invention is characterized in greater detail below by means of the following examples, which are not meant to have any limiting effect on the invention:

### PRODUCTION EXAMPLE 1

| | | |
|---|---|---|
| sodium alginate | 300 | g |
| aluminum chloride | 30 | g |
| water | 12 | L |

### PRODUCTION EXAMPLE 2

| | | |
|---|---|---|
| sodium alginate | 400 | g |
| aluminum sulfate | 50 | g |
| water | 12 | L |

### PRODUCTION EXAMPLE 3

| | | |
|---|---|---|
| sodium alginate | 200 | g |
| apple pectin or | 200 | g |
| citrus pectin | | |
| aluminum chloride | 30 | g |
| water | 12 | L |

### PRODUCTION EXAMPLE 4

| | | |
|---|---|---|
| sodium alginate | 400 | g |
| magnesium chloride | 4 | g |
| aluminum chloride | 20 | g |
| calcium chloride | 10 | g |
| water | 12 | L |

### PRODUCTION EXAMPLE 5

| | | |
|---|---|---|
| sodium alginate | 300 | g |
| aluminum chloride | 30 | g |
| Polysorbate 80 | 20 | g |
| water | 12 | L |

The solutions of the previously mentioned formulations are frozen into plates with a thickness of approximately 4 cm and then dried in a freeze-drier. After drying, the material can be compressed if desired. Then the appropriate administration forms such as tablets or capsules are produced from the plates.

### APPLICATION EXAMPLE

Dried aluminum alginate gels were introduced into synthetic gastric juice and synthetic intestinal juice and studied to determine their dissolution behavior. The inventive aluminum alginate dry gels were insoluble in solutions with a pH in the range of 1.2-4.5. In solutions with a pH of 7, the inventive aluminum alginate dry gels dissolved completely within 30 minutes.

1. Agent for producing a satiety effect and for weight loss consisting of a dried, porous gel or foam of at least one anionic polymer, wherein the agent is present as an aluminum salt.

2. Agent according to claim 1, wherein the agent is present in compressed form.

3. Agent according to claim 1, wherein the agent contains alginate or pectin or a combination thereof as the anionic polymer.

4. Agent according to claim 1, wherein the agent is present as an aluminum alginate, aluminum pectinate, or combination thereof.

5. Agent according to claim 1, wherein the agent also contains active ingredients.

6. Agent according to claim 1, wherein the agent contains vitamins, trace elements, or medicinal compounds as active ingredients.

7. Agent according to claim 1, wherein the agent is administered in the form of tablets, capsules, coated tablets, granulates, or powders.

8. Use of an agent according to claim 1 for the production of a satiety effect and for weight loss.

9. Use of an agent according to claim 1 for regulating the cholesterol balance.

10. Use of an agent according to claim 1 for the production of a composition for producing a satiety effect and for weight loss.

11. Use of an agent according to claim 1 for the production of a composition for regulating the cholesterol balance.
